(19) United States
(12) Patent Application Publication
(10) Pub. No.: US 2004/0142327 A1
(43) Pub. Date: Jul. 22, 2004

(54) Methods for identifying and isolating DNA polymorphisms or genetic markers

(76) Inventor: Jhy-Jhu Lin, Potomac, MD (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/318,399

(22) Filed: Dec. 13, 2002

Related U.S. Application Data
(63) Continuation of application No. 09/585,580, filed on Jun. 2, 2000, now abandoned.
Application No. 09/585,580 is a continuation of application No. 08/975,976, filed on Nov. 21, 1997, now abandoned.
(60) Provisional application No. 60/031,677, filed on Nov. 22, 1996.

(51) Int. Cl. C12Q 1/68; C12P 19/34

(52) U.S. Cl. 435/6; 435/91.2

(57) ABSTRACT

The present invention is related to a method for the identification of specific DNA polymorphisms or genetic markers from a sample of DNA. The invention is particularly related to methods and kits whereby a DNA fragment, found in a first sample of DNA but not in a second sample, may be identified via a series of digestion, amplification, purification, sequencing and detection steps. Most particularly, the invention is directed to a method wherein the detection step comprises transfer of DNA fragments to a solid support and hybridization of the transferred fragments with specific complementary hybridization probes. The invention is further directed to methods for the isolation of a polymorphic DNA fragment or genetic marker identified as described above. This invention has utility in the identification and isolation of cDNA sequences for use in a variety of medical, forensic, industrial and plant breeding procedures employing the methodologies of molecular genetics.

### CROSS REFERENCE TO RELATED APPLICATION

This application claims priority to U.S. Provisional Patent Application No. 60/031,677, filed Nov. 22, 1996, the contents of which are entirely incorporated herein by reference.

### BACKGROUND OF THE INVENTION

1. Field of the Invention

This invention is in the field of molecular and cellular biology. In general, the invention is related to a method for the identification of specific polymorphic genetic sequences or genetic markers from genomic DNA or a cDNA library of an organism. The invention is particularly related to a method whereby a polymorphic DNA fragment or genetic marker, not found in the genome or in cDNA libraries of other tissues from the same or a different organism, may be identified via a series of digestion, amplification, purification, sequencing and detection steps. Most particularly, the invention is directed to a method wherein the detection step comprises transfer of DNA fragments to a solid support and hybridization of the transferred fragments with specific complementary oligonucleotides. This invention has utility in the identification and isolation of polymorphic DNA fragments or genetic markers from genomic DNA or cDNA samples for use in a variety of medical, forensic, industrial and plant breeding procedures employing the methodologies of molecular genetics.

2. Related Art

Genomic DNA

In examining the structure and physiology of an organism, tissue or cell, it is often desirable to determine its genetic content. The genetic framework (i.e., the genome) of an organism is encoded in the double-stranded sequence of nucleotide bases in the deoxyribonucleic acid (DNA) which is contained in the somatic and germ cells of the organism. The genetic content of a particular segment of DNA, or gene, is only manifested upon production of the protein which the gene ultimately encodes. In order to produce a protein, a complementary copy of one strand of the DNA double helix (the “sense” strand) is produced by polymerase enzymes, resulting in a specific sequence of messenger ribonucleic acid (mRNA). This mRNA is then translated by the protein synthesis machinery of the cell, resulting in the production of the particular protein encoded by the gene. There are additional sequences in the genome that do not encode a protein (i.e., “noncoding” regions) which may serve a structural, regulatory, or unknown function. Thus, the genome of an organism or cell is the complete collection of protein-encoding genes together with intervening noncoding DNA sequences. Importantly, each somatic cell of a multicellular organism contains the full complement of genomic DNA of the organism, except in cases of focal infections or cancers, where one or more xenogeneic DNA sequences may be inserted into the genomic DNA of specific cells and not into other, non-infected, cells in the organism. The expression of the genes making up the genomic DNA, however, may vary between individual cells.

cDNA and cDNA Libraries

One common approach to the study of gene expression is the production of complementary DNA (cDNA) clones. In this technique, the mRNA molecules from an organism are isolated from an extract of the cells or tissues of the organism. This isolation often employs solid chromatography matrices, such as cellulose or hydroxyapatite, to which oligomers of thymidine (T) have been complexed. Since the 3′ termini on all eukaryotic mRNA molecules contain a string of adenosine (A) bases, and since A binds to T, the mRNA molecules can be rapidly purified from other molecules and substances in the tissue or cell extract. From these purified mRNA molecules, cDNA copies may be made using the enzyme reverse transcriptase, which results in the production of single-stranded cDNA molecules. The single-stranded cDNAs may then be converted into a complete double-stranded DNA copy of the original mRNA (and thus of the original double-stranded DNA sequence, encoding this mRNA, contained in the genome of the organism) by the action of a DNA polymerase. The protein-specific double-stranded cDNAs can then be inserted into a plasmid, which is then introduced into a host bacterial cell. The bacterial cells are then grown in culture media, resulting in a population of bacterial cells containing (or in many cases, expressing) the gene of interest.

This entire process, from isolation of mRNA to insertion of the cDNA into a plasmid to growth of bacterial populations containing the isolated gene, is termed “cDNA cloning.” If cDNAs are prepared from a number of different mRNAs, the resulting set of cDNAs is called a “cDNA library,” an appropriate term since the set of cDNAs represents the different “volumes” (genes) of functional genetic information present in the source cell, tissue or organism. Genotypic analysis of these cDNA libraries can yield much information on the structure and function of the organisms from which they were derived.

DNA Fingerprinting

To determine the genotype of an organism, tissue or cell, various molecular biological techniques are employed. One such technique often used is known as DNA fingerprinting. This technique relies on the digestion of the DNA of an organism, tissue or cell with a restriction endonuclease enzyme which cleaves the DNA sample into fragments of discrete length. Due to the specificity with which different restriction endonucleases cleave their DNA substrates, a given set of enzymes will always produce the same results, in terms of fragment number and size (the term “size” as used herein is defined as the length and/or molecular weight of a given restriction fragment), from a given DNA sample. The restriction fragments may then be resolved by a variety of techniques such as size exclusion chromatography, gel electrophoresis, or attachment to a variety of solid matrices. Most commonly, gel electrophoresis is performed, and the restriction fragments are resolved into a series of bands on the gel via their differential mobilities within the gel (which is inversely related to fragment size). The pattern of these bands within the gel is specific for a given DNA sample, and is often referred to as the “fingerprint” of that sample.

When the DNA fingerprints of closely related organisms, tissues or even cells are compared, these fingerprints are often quite similar. However, subtle differences between the fingerprints may be observed. These differences, termed “DNA polymorphisms,” or “genetic markers,” tend to increase in number (i.e., the fingerprints become more dissimilar) as DNA samples from more distantly related or unrelated organisms are compared. This technique of examining such Restriction Fragment Length Polymorphisms, or “RFLPs,” has been used for a number of years in genotypic analysis of eukaryotes such as plants (Tanksley, S. D. et al., Bio/Technology 7:257-264 (1989)) and animals, including humans (Botstein, D. et al., Am. J. Hum. Genet. 32:314-331 (1980)). In fact, RFLP analysis is being used in combination with other techniques in molecular biology to determine the complete structure (i.e., the “map”) of the human genome (See, e.g., Donis-Keller, H. et al., Cell 51:319-337 (1987)).

DNA Amplification

One early drawback to the use of RFLP analysis, however, was its requirement for larger amounts of DNA than are typically available in the samples to be analyzed. In addition, complex genomic samples are often difficult to analyze by RFLP, as a multitude of different DNA molecules are simultaneously fragmented and resolved. As a means of overcoming these difficulties, investigators have increasingly turned to methods that increase the copy number of, or “amplify,” specific sequences of DNA in a sample.

A commonly used amplification technique is the Polymerase Chain Reaction (“PCR”) method invented by Mullis and colleagues (U.S. Pat. Nos. 4,683,195; 4,683,202; and 4,800,159). This method uses “primer” sequences which are complementary to opposing regions on the DNA sequence to be amplified. These primers are added to the DNA target sample, along with a molar excess of nucleotide bases and a DNA polymerase (e.g., Taq polymerase), and the primers bind to their target via base-specific binding interactions (i.e., adenine binds to thymine, cytosine to guanine). By repeatedly passing the reaction mixture through cycles of increasing and decreasing temperatures (to allow dissociation of the two DNA strands on the target sequence, synthesis of complementary copies of each strand by the polymerase, and re-annealing of the new complementary strands), the copy number of a particular sequence of DNA may be rapidly increased.

Other techniques for amplification of target nucleic acid sequences have also been developed. For example, Walker et al. (U.S. Pat. No. 5,455,166; EP 0 684 315) described a method called Strand Displacement Amplification (SDA), which differs from PCR in that it operates at a single temperature and uses a polymerase/endonuclease combination of enzymes to generate single-stranded fragments of the target DNA sequence, which then serve as templates for the production of complementary DNA (cDNA) strands. An alternative amplification procedure, termed Nucleic Acid Sequence-Based Amplification (NASBA) was disclosed by Davey et al. (U.S. Pat. No. 5,409,818; EP 0 329 822). Similar to SDA, NASBA employs an isothermal reaction, but is based on the use of RNA primers for amplification rather than DNA primers as in PCR or SDA.

PCR-Based DNA Fingerprinting

Despite the availability of a variety of amplification techniques, most DNA fingerprinting methods rely on PCR for amplification, taking advantage of the well-characterized protocols and automation available for this technique. Examples of these PCR-based fingerprinting techniques include Random Amplified Polymorphic DNA (RAPD) analysis (Williams, J. G. K. et al., Nucl. Acids Res. 18(22):6531-6535 (1990)), Arbitrarily Primed PCR (AP-PCR; Welsh, J., and McClelland, M., Nucl. Acids Res. 18(24):7213-7218 (1990)), DNA Amplification Fingerprinting (DAF; Caetano-Anollés et al., Bio/Technology 9:553-557 (1991)), and microsatellite PCR or Directed Amplification of Minisatellite-region DNA (DAMD; Heath, D. D. et al., Nucl. Acids Res. 21(24):5782-5785 (1993)). All of these methods are based on the amplification of random DNA fragments by PCR, using arbitrarily chosen primers. The utility of these techniques is limited, however, by their extreme sensitivity to the quality of the target DNA, which may be poor in some genomic or cDNA library samples. Use of poor-quality (e.g., fragmented, degraded or otherwise non-intact) DNA in these techniques can lead, for example, to spurious results due to incomplete amplification of desired target DNA sequences.

More recently, a technique named Amplification Fragment Length Polymorphism (AFLP) analysis was developed by Vos and colleagues (EP 0 534 858; Vos, P. et al., Nucl. Acids Res. 23(21):4407-4414 (1995)). This technique, which is also PCR-based, uses specific combinations of restriction endonucleases and adapters of discrete sequences, as well as primers that contain the common sequences of the adapters. In this way, a sequence or fragment of DNA in a complex sample may be specifically amplified and used for further analysis. The value of AFLP in genomic analyses of certain plant and bacterial strains has been demonstrated (Lin, J.-J., and Kuo, J., FOCUS 17(2):66-70 (1995); Lin, J.-J., et al., Plant Molec. Biol. Rep. 14(2):156-169 (1996)), while others have used AFLP for HLA-DR genotyping in humans (Yunis, I. et al., Tissue Antigens 38:78-88 (1991)).

Identification of DNA Polymorphisms or Genetic Markers

Despite the success of genetic mapping using the foregoing techniques, however, these methods are limited in their abilities to identify source-specific DNA sequences (i.e., polymorphic DNA fragments) or genetic markers. This limitation is particularly true for those sequences derived from genomic DNA samples from different cells, tissues or organisms, and for those derived from tissue cDNA libraries which comprise only those DNA molecules that are actively expressed (i.e., used to make proteins) in the particular tissue and which are thus a subset of genomic DNA. For cDNA libraries, however, methods have been developed in the attempt to overcome these limitations.

One such method, termed differential hybridization, relies on the knowledge that specific genes are expressed differentially in certain cells or tissues as opposed to other cells or tissues. To identify these cell- or tissue-specific DNA fragments, one might prepare cDNAs from two different cell or tissue types and separately hybridize the cDNA samples to oligonucleotide probes prepared from each of the samples. The resultant hybridization patterns may then be compared, and any differences observed may indicate the cell- or tissue-specific expression of one or more genes (and thus the presence, in a cDNA library prepared from that cell or tissue, of a specific cDNA). This technique was used to identify growth factor-regulated genes that are specifically expressed in cells stimulated to grow by treatment with serum but that are not expressed in quiescent cells (Lau, H. F., and Nathans, D., EMBO J. 4:3145-3151 (1985)).

An alternative technique for identifying tissue-specific DNAs is the use of subtractive libraries (See Hedrick, S. M. et al., Nature 308:149-153 (1984); Lin et al., FOCUS 14(3):98-101 (1993)). In this method, cDNAs prepared from the one tissue or cell type are mixed with the mRNAs from another, closely related, tissue or cell type. The cDNAs that are expressed in both cells or tissues then form DNA-RNA hybridization complexes, since they are complementary to each other, while the cDNAs expressed selectively in one cell/tissue but not the other will not form such a complex. The DNA-RNA complexes, representing cDNAs that are not tissue-specific, can then be removed from the mixtures (i.e., “subtracted”) by passing the mixture through a poly-dT or hydroxyapatite column, to which the unhybridized cDNAs will not bind. This procedure thus results in a purified sample that is enriched in tissue- or cell-specific cDNAs.

Amplification-Based Cloning

While differential hybridization and the use of subtractive libraries may be suitable for the identification of DNA sequences that are expressed at relatively high levels in the source cells or tissues, they are not particularly useful when the starting samples contain only low levels of genomic DNA (or mRNA used to make cDNAs), or for the identification and isolation of low-copy-number DNA polymorphisms or potential genetic markers. This problem is particularly important when the tissue or cell samples are themselves present in low quantities (as in many medical or forensic applications), or when the specific DNA sequence is expressed at low levels in the cell/tissue samples.

PCR-based cloning of tissue-specific cDNAs has been used in the attempt to overcome the lack of sensitivity of earlier approaches (see, e.g., Lee, C. C., et al., Science 239:1288-1291 (1988)). However, this approach still suffers from the major shortcoming of PCR itself—the requirement for prior knowledge of the nucleotide sequence of the DNA to be amplified, to allow construction of complementary PCR primers. Without knowing the nucleotide sequence of the target DNA, PCR cannot be performed in order to amplify this sequence in the sample. Since the target sequences are not known in many medical or forensic samples, PCR-based cloning is not useful for the identification or isolation of tissue-specific cDNAs from these samples. For the same reasons, these techniques are not suitable for the identification of previously unknown or uncharacterized genes from cDNA libraries or genomic samples. Furthermore, as noted above, the complexity of genomic DNA limits the utility of these techniques in the identification and isolation of genetic markers from the genome of a cell or organism.

These PCR-based techniques have an additional shortcoming—the need for detectably labeled primer oligonucleotides to provide a means for detecting the RFs, usually by gel electrophoresis and autoradiography. This method is often disadvantageous as it (a) employs radioisotopes; (b) has a lack of sensitivity; and (c) is limited as to molecules that are suitable as “probes.” Furthermore, when labeled primers are used to conduct amplification, only the fragments that are amplified may be subsequently analyzed, providing at least two further limitations: (a) a small polymorphic DNA fragment or genetic marker (e.g., a minisatellite sequence) contained within a larger fragment that is not specific to a single DNA sample will not be detected; and (b) a polymorphic DNA fragment or genetic marker that is the same size (i.e., molecular weight or nucleotide base length) as another amplified nonpolymorphic DNA fragment will colocalize in electrophoresis gels with the latter and will thus not be detected.

Thus, there remains an unmet need for a rapid, reproducible and reliable technique for identifying polymorphic DNA fragments or potential genetic markers that are unique to the genomes or cDNA libraries of specific organisms, tissues or cells, without prior knowledge of the nucleotide sequence of the unique DNA fragments. Particularly desirable is a technique that would rapidly identify specific DNA sequences found in one source genome or library but not in another. Also particularly desirable is a method that obviates the use of detectably labeled primers during the amplification process, such that detection of the molecular markers proceeds independently of the specific primers used, and such that polymorphic DNA fragments or genetic markers are more sensitively detected. Such a technique would find utility in a variety of applications, particularly in clinical, forensic, industrial and plant breeding settings.

### BRIEF SUMMARY OF THE INVENTION

The present invention is directed to an AFLP-based technique that addresses this unmet need. The invention is particularly directed to a method for identifying a polymorphic DNA fragment or a genetic marker in a one sample of DNA which is not present in another sample of DNA. This method comprises (a) digesting the samples of DNA with at least one restriction enzyme to give a collection of restriction fragments; (b) amplifying the collection of restriction fragments; (c) separating the restriction fragments according to size, which as used herein is defined as the length and/or molecular weight of the restriction fragments; (d) transferring the separated fragments to a solid support, which is preferably a nylon membrane or a nitrocellulose membrane; (e) contacting the immobilized restriction fragments with one or more hybridization probes complementary to one or more of the immobilized restriction fragments under conditions stringent for hybridization of the probes to the immobilized restriction fragments; and (f) comparing the pattern of restriction fragments from one sample of DNA to the pattern of fragments from the other sample of DNA. This aspect of the invention may further comprise sequencing the unique polymorphic DNA fragment or genetic marker. In another aspect of the invention, the oligonucleotide probe comprises a telomere repeat sequence. In another aspect of the invention, the hybridization probes are distinctly and detectably labeled. The invention is also directed to such a method further comprising removing the hybridization probe from the solid support; contacting the immobilized restriction fragments with a second hybridization probe; and comparing the pattern of restriction fragments from one sample of DNA to the pattern of fragments from the other sample of DNA. The present invention also encompasses the above method which further comprises isolating at least one polymorphic DNA fragment or genetic marker and inserting the polymorphic DNA fragment or genetic marker into a vector, which may be an expression vector, for use in transfecting or transforming a prokaryotic or eukaryotic host cell. The polymorphic DNA fragment or genetic marker may be amplified prior to insertion into the vector. In another aspect of this embodiment, the polymorphic DNA fragment or genetic marker may be sequenced according to routine nucleic acid sequencing methods.

In a further embodiment, the present invention provides a method for isolating a polymorphic DNA fragment or genetic marker from a sample of DNA. This method comprises (a) mixing one or more of the unique fragments identified as summarized above, or one or more oligonucleotide probes which are complementary to the fragments, with a sample of DNA, derived from a genomic DNA source or a cDNA library source, under conditions stringent for hybridization of the unique fragments or oligonucleotide probes to the sample of DNA; and (b) isolating a polymorphic DNA fragment or genetic marker which is complementary to the unique fragments or to the oligonucleotide probes. According to this aspect of the present invention, the isolation may be accomplished by gel electrophoresis, density gradient centrifugation, sizing chromatography, affinity chromatography, immunoadsorption, or immunoaffinity chromatography. In this embodiment, the isolated polymorphic DNA fragment or genetic marker may also be sequenced, amplified, or inserted into a vector (which may be an expression vector). Polymorphic DNA fragments or genetic markers isolated by this embodiment of the present invention will be useful in, for example, the preparation of DNA or RNA probes, and to aid in the medical and forensic applications described above.

The invention also encompasses the methods described above, wherein the amplification of the polymorphic DNA fragment or genetic marker is accomplished by a method comprising (a) ligating one or more adapter oligonucleotides to a DNA molecule comprising a polymorphic DNA fragment or genetic marker to form a DNA-adapter complex; (b) hybridizing the DNA-adapter complex, under stringent conditions, with one or more oligonucleotide primers which are complementary to the adapter portion of the DNA-adapter complex to form a hybridization complex; and (c) amplifying the DNA-adapter complex. In this aspect of the invention, the adapter oligonucleotide may contain one or more restriction sites which may be used to insert the DNA-adapter complex into a vector.

According to the present invention, the first and second samples of DNA may be derived from the genome of, or a cDNA library prepared from, an individual cell (which may be prokaryotic or eukaryotic), a tissue (which may be a plant or an animal tissue, most preferably a human tissue including a human embryonic or fetal tissue), an organ, or a whole organism. The molecular marker or polymorphic DNA fragment or genetic marker identified according to this embodiment of the invention may be a cancer marker, an infectious disease marker, a genetic disease marker, a marker of embryonic development or an enzyme marker. In one aspect of the invention, one sample of DNA may be derived from a sample from an animal suffering from an infectious disease (e.g., a disease of bacterial, fungal, viral or parasitic origin) and the other sample may be from an animal not suffering from an infectious disease. In another aspect, one sample of DNA may be derived from an animal suffering from cancer and the other may be derived from an animal not suffering from cancer. In another aspect, one sample of DNA may be obtained from a cancerous animal tissue and another sample may be obtained from a noncancerous animal tissue, which tissues may both be obtained from the same animal. In another aspect, one sample of DNA may be from an animal suffering from a genetic disease and the other sample of DNA may be from an animal not suffering from a genetic disease. In another aspect, one sample of DNA may be derived from a diseased plant and the other sample may be derived from a non-diseased plant. In another aspect, one sample of DNA may be from a plant resistant to an environmental stress, which may be drought, excess temperature, diminished temperature, chemical toxicity by herbicides, pollution, excess light or diminished light, and the other sample may be from a plant not resistant to an environmental stress. In another aspect, one sample of DNA may be from a first strain or variety of an organism and the other sample of DNA may be from a second strain or variety of the organism. In another aspect, one sample of DNA may be from a pathogenic microorganism and the other sample of DNA may be from a non-pathogenic microorganism.

In another preferred embodiment, the present invention provides a method of determining the relationship between a first individual and a second individual comprising (a) digesting samples of DNA obtained from the first and second individuals with at least one restriction enzyme to give a collection of restriction fragments; (b) separating the restriction fragments from the first and second individuals according to size; (c) immobilizing the separated restriction fragments on a solid support, which is preferably a nylon or nitrocellulose membrane; (d) contacting the immobilized restriction fragments with one or more hybridization probes complementary to one or more of the immobilized restriction fragments under conditions stringent for hybridization of the probes to the immobilized restriction fragments; and (e) determining the similarities and dissimilarities of the sizes or concentrations of the restriction fragments separated in (b). In a preferred aspect of this embodiment, the oligonucleotide comprises a telomere sequence. In another preferred aspect of this embodiment, the hybridization probes are distinctly and detectably labeled.

In another preferred embodiment, the invention is directed to a kit used for the identification and isolation of a polymorphic DNA fragment or genetic marker. A kit according to this aspect of the invention may comprise a carrier means having in close confinement therein two or more container means, wherein a first container means contains an oligonucleotide adapter DNA molecule and a second container means contains a DNA polymerase enzyme, preferably a thermostable DNA polymerase enzyme, and most preferably Taq DNA polymerase, Tne DNA polymerase or Tma DNA polymerase, or mutants or derivatives thereof A kit according to this aspect of the invention may further comprise one or more additional container means, wherein a first additional container means contains a hybridization probe and/or a restriction endonuclease.

Other preferred embodiments of the present invention will be apparent to one of ordinary skill in light of the following drawings and description of the invention, and of the claims.

### BRIEF DESCRIPTION OF THE FIGURES

FIGS. 1A and 1B are photographs showing a comparison of AFLP patterns generated by radiolabeled AFLP primers and autoradiography (1A) and by membrane blotting/hybridization using enzyme-labeled AFLP hybridization probes and chemiluminescence photography or luminography (1B). DNA samples were prepared from 16 different strains of Agrobacterium tumefaciens: Lane 1, K84; Lane 2, A4; Lane 3, 15834; Lane 4, AG44; Lane 5, AB2/73; Lane 6, pp-1; Lane 7, Rm1021; Lane 8, C58; Lane 9, AB4; Lane 10, S21; Lane 11, Ag87; Lane 12, Ag3; Lane 13, A136; Lane 14, Tm4; Lane 15, S4; Lane 16, LBA4404.

FIGS. 2A and 2B are luminographs demonstrating AFLP polymorphisms detected in different strains of Collectotrichum (FIG. 2A) and soybean (FIG. 2B) by membrane blotting/hybridization using enzyme-labeled AFLP hybridization probes and chemiluminescence. Collectotrichum strains (FIG. 2A): Lane 1, Ari-NW; Lane 2, S1-1; Lane 3, 586; Lane 4, 486, Lane 5, 1418(8); Lane 6, 683(Lc); Lane 7, S7RR; Lane 8, C129; Lane 9, 662(10); Lane 10, C9. Soybean strains (FIG. 2B): Lanes 1, 3, 5 and 7: Noric; Lanes 2, 4, 6 and 8: BARC. Soybean adapters (FIG. 2B): Lanes 1, 2: MseI+CGA (SEQ ID NO:3); Lanes 3, 4: MseI+CGC (SEQ ID NO:4); Lanes 5, 6: MseI+CGG (SEQ ID NO:5); Lanes 7, 8: MseI+CGT (SEQ ID NO:6).

FIGS. 3A and 3B are autoradiograms demonstrating AFLP polymorphisms detected in different strains of peach by membrane blotting/hybridization using enzyme-labeled hybridization probes comprising telomere repeat sequences (FIG. 3A) or by standard AFLP analysis using ³β-labeled primers (FIG. 3B). Strains: Lanes 1, 3, 5, 7: Bologna Pillar; Lanes 2, 4, 6, 8: Flavortop.

### DETAILED DESCRIPTION OF THE INVENTION

The present invention provides a method for identifying and isolating a molecular marker comprising a polymorphic DNA fragment or genetic marker from a sample of genomic DNA or from a cDNA library. It will be readily appreciated by those skilled in the art that using the methods of this invention, any DNA fragment comprising a sequence of contiguous nucleotide bases that is specifically contained within a given genome or cDNA library may be identified and isolated.

Sources of DNA

Suitable sources of DNA for the preparation of genomic DNA or cDNA libraries, including a variety of cells, tissues, organs or organisms, may be obtained through any number of commercial sources (including American Type Culture Collection (ATCC), Rockville, Md.; Jackson Laboratories, Bar Harbor, Me.; Cell Systems, Inc., Kirkland, Wash.; Advanced Tissue Sciences, La Jolla, Calif.). Cells that may be used as starting materials for DNA preparation may be prokaryotic (bacterial, including members of the genera Escherichia, Serratia, Salmonella, Staphylococcus, Streptococcus, Clostridium, Chlamydia, Neisseria, Treponema, Mycoplasma, Borrelia, Bordetella, Legionella, Pseudomonas, Mycobacterium, Helicobacter, Agrobacterium, Rhizobium, and Streptomyces) or eukaryotic (including fungi such as Collectotrichum or yeasts, plants, protozoans and other parasites, and animals including humans and other mammals). Any mammalian somatic cell may be used for preparation of genomic DNA, since each somatic cell in an organism contains a full complement of the organism's genomic DNA. Particularly suitable as cellular sources of genomic DNA are blood cells (erythrocytes and leukocytes), endothelial cells, epithelial cells, neuronal cells (from the central or peripheral nervous systems), muscle cells (including myocytes and myoblasts from skeletal, smooth or cardiac muscle), connective tissue cells (including fibroblasts, adipocytes, chondrocytes, chondroblasts, osteocytes and osteoblasts) and other stromal cells (e.g., macrophages, dendritic cells, Schwann cells), although as noted above other somatic cells, including the progenitors, precursors and stem cells that give rise to the above-described somatic cells, are equally suitable. Mammalian germ cells (spermatocytes and oocytes) may also be used for the preparation of cDNA libraries, as may the progenitors, precursors and stem cells that give rise to the above somatic and germ cells, although these cells are not favored for the preparation of genomic DNA since they comprise only half the complement of genomic DNA (i.e., “1N”) of the organism that is found in somatic cells. Also suitable for use in the preparation of DNA are mammalian tissues or organs such as those derived from brain, kidney, liver, pancreas, blood, bone marrow, muscle, nervous, skin, genitourinary, circulatory, lymphoid, gastrointestinal and connective tissue sources, as well as those derived from a mammalian (including human) embryo or fetus. These cells, tissues and organs may be normal, or they may be pathological such as those involved in infectious diseases (caused by bacteria, fungi or yeast, viruses (including AIDS) or parasites), in genetic or biochemical pathologies (e.g., cystic fibrosis, hemophilia, Alzheimer's disease, schizophrenia, muscular dystrophy or multiple sclerosis), or in cancerous processes.

AFLP Analysis

Purification of DNA

Once the starting cells, tissues, organs or other samples are obtained, genomic DNA may be prepared therefrom by methods that are well-known in the art (See, for example, Sambrook, J., et al., Molecular Cloning: A Laboratory Manual, 2nd ed., Cold Spring Harbor, N.Y.: Cold Spring Harbor Laboratory Press, pp. 9.16-9.23 (1989); Kaufman, P. B., et al., Handbook of Molecular and Cellular Methods in Biology and Medicine, Boca Raton, Fla.: CRC Press, pp. 1-26 (1995)). Similarly, cDNA libraries may be prepared from the samples by methods that are well-known in the art (See, for example, Maniatis, T. et al., Cell 15:687-701 (1978); Okayama, H., and Berg, P., Mol. Cell. Biol. 2:161-170 (1982); Gubler, U., and Hoffman, B. J., Gene 25:263-269 (1983)). The DNA samples thus prepared may then be used to identify and isolate unique polymorphic DNA fragments or genetic markers by the present invention.

Identification of Polymorphic DNA Fragments or Genetic Markers

Purified DNA may be examined by AFLP for identification of specific polymorphic DNA fragments or genetic markers according to the present invention. AFLP was originally developed as a method for DNA fingerprinting analysis of bacterial, yeast, plant and animal cells (EP 0 534 858; Vos, P. et al., Nucl. Acids Res. 23(21):4407-4414 (1995); Lin, J.-J., and Kuo, J., Focus 17(2):66-70 (1995)). In the present invention, the AFLP technique has been modified to provide, in a first preferred embodiment, a method for identifying a polymorphic DNA fragment or genetic marker from a sample of DNA and, in a second preferred embodiment, a method for isolating a polymorphic DNA fragment or genetic marker from a sample of DNA.

AFLP may be carried out using a commercially available system such as the AFLP Analysis System I (Life Technologies, Inc; Rockville, Md.) which contains a detailed methods manual, the disclosure of which is fully incorporated herein by reference. Alternatively, AFLP analysis may be performed using a combination of materials and methods that are modified from those commonly used in the art (EP 0 534 858; Vos, P. et al., Nucl. Acids Res. 23(21):4407-4414 (1995); Lin, J.-J., and Kuo, J., Focus 17(2):66-70 (1995)).

The power of the AFLP technique is based on its use of generic primers and “adaptors” which allow amplification of DNA fragments or genetic markers without any prior knowledge of the nucleotide sequences of those fragments. As used herein, the term “adaptor” refers to a nucleic acid molecule comprising a sequence of 2-8 contiguous nucleotides comprising the nucleotide sequences at the restriction sites in the target DNA samples; an individual adaptor will thus have a nucleotide sequence complementary to that of the cut ends of the RFs. In this way, the AFLP-based method of the present invention is more useful for identification of previously unknown polymorphic DNA fragments or genetic markers in DNA samples than is traditional PCR, which requires prior knowledge of the nucleotide sequence of the target DNA fragment or genetic marker in order to design appropriate amplification primers.

In the initial step of AFLP, purified DNA is digested with a panel of enzymes usually containing two restriction enzymes. Ordinarily, the two restriction enzymes have sequence specificities sufficiently different from one another so as to prevent overlap of digestion (and thus over-degradation) of the target DNA sequences. For example, the enzymes EcoRI and MseI may be used in combination to digest target DNA, as the restriction site specificities of these two enzymes are significantly different. However, other combinations of restriction enzymes may be used in carrying out the present invention with equal likelihood of success.

Once the DNA sample has been digested with restriction enzymes (producing a collection of “restriction fragments,” hereinafter referred to as “RFs”), the resultant RFs are ligated with adaptor nucleotide sequences. The use of adaptors is necessary since after digestion, the cut ends of the RFs, to which the PCR primers will bind, are often too short for optimal binding of the primers. Ligation of adaptor nucleotide sequences to the cut ends of the RFs thus extends the length of these primer binding sites, improving the efficiency of primer binding and thus of amplification. The nucleotide sequences of these adaptors are chosen so as to contain the nucleotide sequences at the restriction sites in the target DNA samples. The adaptors usually will have a stretch of 2-8 contiguous nucleotides which are complementary to the cut ends of the RFs; thus, the adaptors bind to the RFs via normal DNA base-pairing and thereby extend the terminal sequence of the RFs.

Once the adaptors have been ligated to the DNA RFs, the fragments are amplified via PCR according to standard methods used for cDNA fragment amplification (Lin and Kuo, Id), using PCR primer oligonucleotides that hybridize to the extended ends of the RFs (i.e., the binding regions of the primers are complementary to the sequences of the adaptors) under conditions used for PCR. This approach provides the additional advantage that the actual sequences of the genomic DNA fragments that are the targets for amplification need not be known, since the primers are designed to be specific for a restriction site rather than a particular gene. Accordingly, generic primers may be used, with their nucleotide sequences being dependent upon the combination of restriction enzymes used to digest the target DNAs (Vos et al, Id.; Lin and Kuo, Id.). For example, the EcoRI primer will contain the sequence of the EcoRI restriction site (underlined below) coupled to a core sequence and an arbitrary extender of three-base repeat units:

| | |
|---|---|
| 5′-GAC TGC GTA CCA ATT C-3′ | (SEQ ID NO: 1) |
| EcoRI + 0 primer | |

Similarly, the MseI primer will contain the nucleotide sequence of the MseI restriction site linked to different core and extender sequences:

| | |
|---|---|
| 5′-GAT GAG TCC TGA GTA A-3′ | (SEQ ID NO: 2) |
| MseI + 0 primer | |

Ordinarily, these AFLP-specific primers are detectably labeled, thereby providing a means for subsequent identification of the polymorphic DNA fragments or potential genetic markers. In the present invention, however, labeling of the primers is not necessary; polymorphic DNA fragments or genetic markers are identified instead by immobilization of separated DNA fragments on a solid support and subsequent hybridization with oligonucleotide probes, which may be detectably labeled, as described below. Accordingly, the present invention preferably uses unlabeled or “cold” primers in the amplification of the RFs.

Following amplification, the samples are prepared for separation of the DNA fragments, a procedure which permits the determination of the presence of specific polymorphic DNA fragments or genetic markers in the DNA samples. The amplified restriction fragments may be separated by any physical or biochemical means including gel electrophoresis, chromatography (including sizing, affinity and immunochromatography), density gradient centrifugation and immunoadsorption. For carrying out the present invention, separation of DNA fragments by gel electrophoresis is particularly preferred, as it provides a rapid and highly reproducible means of sensitive separation of a multitude of DNA fragments, permits direct comparison of the fragments in several samples of DNA simultaneously, and facilitates subsequent transfer of the DNA fragments to a solid support in preparation for detection by oligonucleotide hybridization.

Gel electrophoresis is typically performed on agarose or polyacrylamide sequencing gels according to standard protocols (Lin, J.-J., and Kuo, J., FOCUS 17(2):66-70 (1995)), preferably using gels containing polyacrylamide at concentrations of 3-8% and most preferably at about 5%, and containing urea at a concentration of about 8M. Samples are loaded onto the gels, usually with samples containing DNA fragments prepared from different sources being loaded into adjacent lanes of the gel to facilitate subsequent comparison.

Following electrophoretic separation, the DNA fragments are immobilized onto a solid support. Solid supports that are preferable for use in the present invention are membrane filters, most preferably nylon or nitrocellulose membrane filters. Transfer of separated DNA fragments from the gel to the solid support may be effected by electrophoretic or pressure blotting techniques which are routine in the art (see, e.g., Southern, E. M., J. Mol. Biol. 98:503-517 (1975); Watson, J. D., et al., in: Recombinant DNA, 2nd Ed., New York: Scientific American Books, pp. 127-130 (1992)).

Once immobilized on a solid support, the DNA fragments may be analyzed for the presence of polymorphic DNA fragments or potential genetic markers by hybridization with specific hybridization probes, using techniques that are well-known and routine to one of ordinary skill in the art (see, e.g., Southern, E. M., Id.; Watson, J. D., et al., Id.; Sambrook, J., et al., Molecular Cloning: A Laboratory Manual, 2nd ed., Cold Spring Harbor, N.Y.: Cold Spring Harbor Laboratory Press, pp. 9.47-9.57 (1989)). As used herein, the term “hybridization probe” refers to a DNA molecule comprising a contiguous sequence of nucleotides that is substantially complementary to one or more of the immobilized DNA fragments. By “substantially complementary” is meant that the nucleotide sequences have a sequence identity of at least 70%, preferably at least 85%, more preferably at least 95%, still more preferably at least 96%, still more preferably at least 97%, still more preferably at least 98%, and most preferably at least 99%. The hybridization probes used in the methods of the present invention are preferably oligonucleotides, which may be directed to the AFLP adapter sequence, to the primer sequence, to a different known or previously characterized nucleotide sequence (e.g., a telomere repeat or minisatellite sequence), to a degenerate DNA sequence, or to a random nucleotide sequence. Hybridization probes used in the present methods may be derived from natural sources, e.g., a genomic DNA sample, or from synthetic sources, e.g., a cDNA library or a synthetic oligonucleotide.

In the present invention, the solid support comprising the immobilized DNA fragments is contacted with the hybridization probe under conditions stringent for hybridization of the probe to the immobilized DNA fragments. As used herein, the term “conditions stringent for hybridization” is defined, as is generally understood to one of ordinary skill in the art, as incubation of the DNA fragments with the hybridization probe(s) for 18-24 hours at about 42° C. in a solution comprising 5×SSC (150 millimolar sodium chloride, 15 millimolar trisodium citrate), 50 millimolar sodium phosphate (pH about 7.6), 5× Denhardt's solution, 50% formamide, 10% dextran sulfate and 20 grams per milliliter denatured, sheared salmon sperm DNA. In the present invention, the oligonucleotides used as hybridization probes may be detectably labeled, preferably with an enzyme (such as alkaline phosphatase or peroxidase), a radioisotope (such as ³²P, ³³P, ³H or ¹⁴C), a vitamin (such as biotin), a vitamin-binding protein (such as avidin or streptavidin), or a fluorescent molecule (such as fluorescein or rhodamine); most preferably, the oligonucleotides are detectably labeled with an enzyme such as alkaline phosphatase.

Following hybridization, the polymorphic DNA fragments or genetic markers may be visualized and identified by a variety of techniques that are routine to those of ordinary skill in the art. In a first such technique, the blot is air-dried and exposed to X-ray film (for detection of radioisotopes). In an alternative technique, the blot is air-dried and developed using the specific enzyme-cofactor-substrate combinations that are optimal for the enzyme, cofactor, vitamin, vitamin-binding protein or other label used to detectably label the hybridization probes. For example, blots in which the oligonucleotides are labeled with alkaline phosphatase may be developed using a combination of nitroblue tetrazolium chloride (NBT) and 5-bromo-4-chloro-3-indolylphosphatep-toluidine salt (BCIP), which produce a dark blue/black colored precipitate in the location of the DNA fragment/oligonucleotide hybridization complex on the solid support. Alternatively, the enzyme may be used to cleave a substrate to yield a fluorescent, phosphorescent, or chemiluminescent product; such substrates are available, for example, from Sigma (Saint Louis, Mo.) or Tropix (Bedford, Mass.). Other enzyme-cofactor-substrate combinations appropriate for use in the present invention are routine and well-known to one of ordinary skill in the art. Following color development (for visible substrates) or fluorescent, phosphorescent or chemiluminescent development, the blot may be photographed using standard photographic films and techniques, preferably with a technical grade film such as Bio-Max MR or Tri-X Pan Kodak; Rochester, N.Y.).

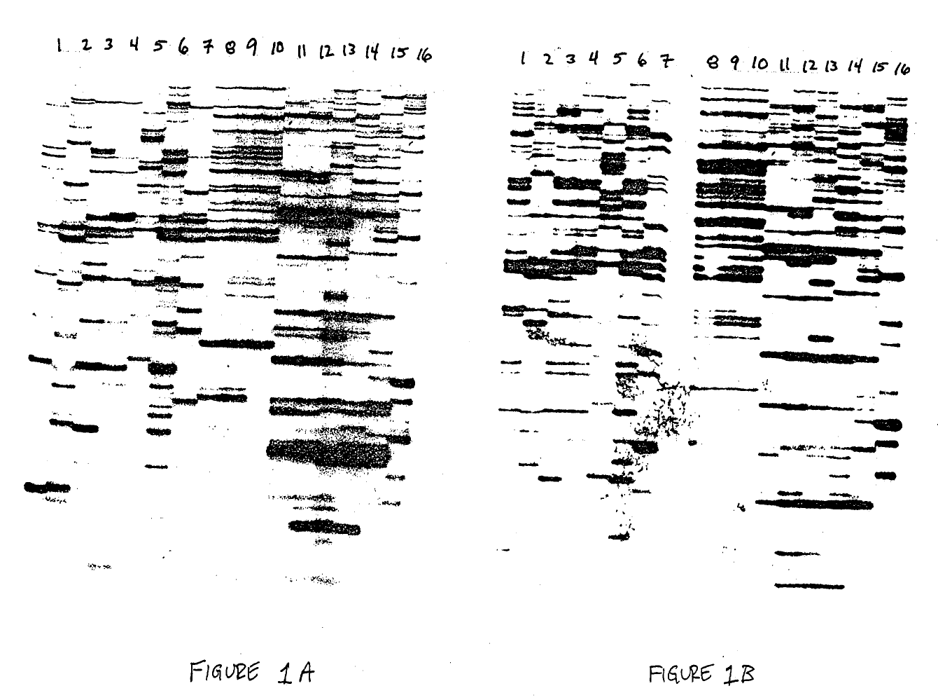

After development, the film is examined for the pattern of bands in each lane of the gel, each band corresponding to a different DNA species or fragment (see FIGS. 1-3). The migration of DNA fragments within the gel is proportional to their size (length and/or molecular weight)—i.e., larger fragments migrate more slowly (and thus form bands closer to the top of the gel), while smaller fragments migrate more quickly (and thus form bands closer to the gel bottom). One can thus examine the films for the presence of one or more unique bands in one lane of the gel; the presence of a band in one lane (corresponding to a single sample, cell or tissue type, or strain of organism) that is not observed in other lanes indicates that the DNA fragment comprising that unique band is source-specific and thus a potential genetic marker. Moreover, the methods of the present invention make possible the detection of polymorphic DNA fragments or genetic markers that may not be otherwise detected. Included in this category are polymorphic DNA fragments or genetic markers that are contained within a larger non-polymorphic fragment, and fragments that are the same size (i.e., molecular weight or length) as one or more amplified non-polymorphic DNA fragments which may co-localize with the latter in electrophoretic gels. Thus, the present invention provides a sensitive method of detecting novel DNA polymorphisms or potential genetic markers that other approaches might not identify.

As an alternative approach, following hybridization and analysis with a first hybridization probe the blot may be washed to remove the hybridization probe and then reprobed with a second hybridization probe. Methods for removing hybridization probes from solid supports such as nitrocellulose or nylon membranes, in preparation for analysis with a second probe, are well known to one of ordinary skill in the art and are described in detail, for example, in Sambrook, J., et al., Molecular Cloning: A Laboratory Manual, 2nd ed., Cold Spring Harbor, N.Y.: Cold Spring Harbor Laboratory Press, p. 9.58 (1989). Since drying of the solid support irreversibly binds the hybridization probes (Id), and since typical autoradiography protocols require drying of the support prior to its exposure to film (as described above), washing and reprobing of the solid support is preferably done when detecting the presence of DNA polymorphisms or potential genetic markers using hybridization probes labeled with other than radioisotopes, e.g., with enzymes, fluorescent molecules or other labels which may be “developed” without drying the solid support. Using these approaches, a single blot may be probed, washed, and reprobed through multiple cycles with a battery of hybridization probes, to yield substantial information about the presence of a multitude of potential DNA polymorphisms or potential genetic markers in a single sample of DNA. Such an approach not only expands the capabilities of amplification-based detection methods, but also provides substantial savings in starting materials, reagents and time.

By the methods of the present invention, such multiple determinations may alternatively be accomplished by simultaneously probing the solid support with multiple hybridization probes, each of which may be distinctly and detectably labeled. For example, a single blot may be probed at once with three separate oligonucleotide probes—one labeled with fluorescein, a second with rhodamine, and a third with phycocyanin. The blot may then be examined using methods for detecting fluorescence (e.g., a fluorescence microscope equipped with appropriate filters) to rapidly and concurrently determine the presence and localization of polymorphic DNA fragments or genetic markers targeted by the individual probes. This approach could also be combined with the “probe-wash-reprobe” method described above to provide even more rapid and efficient screening of a DNA sample for the presence of a variety of DNA polymorphisms and potential genetic markers.

Isolation and Characterization of Polymorphic DNA Fragments or Genetic Markers

A variety of polymorphic DNA fragments or potential genetic markers can thus be identified using the methods of the present invention by comparing the pattern of bands on the films depicting various samples. One can extend this approach, in a second preferred embodiment, to isolate and characterize these polymorphic DNA fragments or genetic markers. In this second embodiment, a duplicate gel is run, comprising the same samples separated in the identification steps described above. After identification, one or more of the polymorphic DNA fragments or genetic markers may be localized in, and removed from, the second gel. Localization of these polymorphic DNA fragments or genetic markers in the second gel is effected by overlaying the developed hybridization blot, or film record thereof, directly over the second gel, thus allowing the blot or film to be used as a guide or template to localize the DNA fragments of interest in the second gel. Removal of these fragments from the gel may be effected by a number of means including electroelution, or preferably by physical excision wherein the fragments represented by unique bands on the developed blot or film may then be carefully cut from the second gel through the corresponding band on the blot or film using, for example, a scalpel, razor or scissors. The DNA may then be eluted from the gel by incubating the slice for about 18-24 hours at 37° C. in a buffered salt solution, such as TE buffer (50 mM glucose, 50 mM TRIS®-HCl, 10 mM EDTA, pH 8.0). Following elution, the DNA sample in TE buffer may be loaded into a syringe containing sterilized glass wool and filtered through the glass wool into a sterile tube via centrifugation at about 250-500×g for about 10 minutes at about 20-25° C. Alternatively, this filtration may be accomplished via other chromatographic methods that are well known in the art, such as using standard glass wool columns and peristaltic pumping. After being filtered through glass wool, the DNA-containing sample is filtered through a desalting/buffer exchange column (e.g., using SEPHADEX® or a pre-packed PD-10 column available from Pharmacia, Piscataway, N.J.) according to the manufacturer's instructions. This desalting/buffer exchange step may be accomplished by other methods that are routine in the art, e.g., via batch dialysis, although the use of columns for this purpose overcomes the longer time required, higher cost and sample loss that often accompany standard dialysis methods. The isolated polymorphic DNA fragments or genetic markers may then be eluted from the desalting column in deionized, distilled water and lyophilized and stored at 4° C. to −70° C. until use. Alternatively, these AFLP-defined polymorphic DNA fragments or genetic markers can be immediately dissolved in TE buffer and re-amplified as outlined above to increase their concentration. Prior to or following this amplification, the polymorphic DNA fragments or genetic markers may be inserted into standard nucleotide vectors suitable for transfection or transformation of a variety of prokaryotic (bacterial) or eukaryotic (yeast, plant or animal including human and other mammalian) cells. Vectors suitable for these purposes, and methods for insertion of DNA fragments therein, will be well-known to one of ordinary skill in the art.

Kits

The invention also provides kits for use in the identification and isolation of a polymorphic DNA fragment according to the present methods. Kits according to the present invention may comprise a carrying means being compartmentalized to receive in close confinement therein two or more containers such as vials, tubes, bottles and the like. Each of such containers may comprise components or a mixture of components needed to perform DNA amplification or analysis.

A kit for identifying a polymorphic DNA fragment may comprise a number of containers. A first container may, for example, contain an oligonucleotide adapter DNA molecule. A second container may contain a DNA polymerase, preferably a thermostable DNA polymerase, and most preferably Taq DNA polymerase, Tne DNA polymerase, Tma DNA polymerase, or mutants or derivatives thereof. Taq DNA polymerase is commercially available, for example from Life Technologies, Inc. (Rockville, Md.), or may be isolated from its natural source, the thermophilic bacterium Thermus aquaticus, as described previously (U.S. Pat. Nos. 4,889,818 and 4,965,188). Tne DNA polymerase may be isolated from its natural source, the thermophilic bacterium Thermotoga neapolitana (See WO 96/10640), and Tma DNA polymerase from its natural source, the thermophilic bacterium Thermotoga maritima (See U.S. Pat. No. 5,374,553). Methods for producing mutants and derivatives of thermophilic DNA polymerases, particularly of Tne and Tma polymerases, are disclosed in co-pending U.S. patent application Ser. No. 08/689,818, filed Sep. 6, 1996, and co-pending U.S. patent application Ser. No. 08/689,807, filed Sep. 6, 1996, which are incorporated by reference herein in their entirety.

Kits according to the present invention may also comprise one or more container means in addition to those described above. The additional container means of such kits may contain, for example, a hybridization probe which may be detectably labeled, a restriction endonuclease, and/or other components to be used in the identification and isolation of polymorphic DNA fragments or genetic markers according to the present invention, such as polyacrylamide, agarose, urea, buffer salts, detergents and the like.

Of course, it is also possible to combine one or more of these reagents in a single tube. A detailed description of such formulations at working concentrations is described in co-pending U.S. application Ser. No. 08/801,720, filed on Feb. 14, 1997, which is incorporated by reference herein in its entirety.

Use of Polymorphic DNA Fragments or Genetic Markers

The polymorphic DNA fragments or potential genetic markers that are identified and isolated by the methods and kits of the present invention may be further characterized, for example by cloning and sequencing (i.e., determining the nucleotide sequence of the polymorphic DNA fragments or genetic markers), by methods described above and others that are standard in the art (see also U.S. Pat. Nos. 4,962,020 and 5,498,523, which are directed to methods of DNA sequencing). Alternatively, these polymorphic DNA fragments or genetic markers may be used for the manufacture of various materials in industrial processes, such as hybridization probes or therapeutic proteins (dependent upon transcription and translation of the DNA fragments or genetic markers, or the production of synthetic peptides or proteins with amino acid sequences deduced from the nucleotide sequences of the specific polymorphic DNA fragments or genetic markers) by methods that are well-known in the art. Production of hybridization probes from polymorphic DNA fragments or genetic markers will, for example, provide the ability for those in medical fields to examine a patient's cells or tissues for the presence of a particular polymorphism which may serve as a marker of cancer or of an infectious or genetic disease, as a marker of embryonic development, as a tissue-specific marker or as an enzyme marker. Particularly suitable for identification by the methods of the present invention are polymorphic DNA fragments or genetic markers which may serve as markers of genetic diseases such as cystic fibrosis, hemophilia, Alzheimer's disease, schizophrenia, muscular dystrophy or multiple sclerosis. Also suitable for identification by the methods of the present invention are polymorphic DNA fragments or genetic markers which may serve as genetic markers associated with pathogenicity (e.g., virulence genes) of microorganisms. Hybridization probes prepared by the methods of the present invention can further be used to isolate DNA fragments or genetic markers from other cells, tissues or organisms for further characterization. In this application, hybridization probes comprising the AFLP-defined polymorphic DNA fragments or genetic markers identified above, or one or more oligonucleotide probes complementary to these fragments, are hybridized under conditions stringent for hybridization (as defined above) with a sample of DNA isolated from genomic DNA of, or a cDNA library prepared from, a cell, tissue or organism. Following hybridization, the samples may be washed in 0.1×SSC at about 65° C. to further reduce nonspecific background, and the unique genomic DNA or cDNA fragments so isolated may be amplified and characterized as described above. Together, these abilities will assist medical professionals and patients in diagnostic and prognostic determinations as well as in the development of treatment and prevention regimens for these and other disorders. Furthermore, such methods will be useful to those involved in plant breeding, in the identification of new strains, varieties or cultivars of plants, or in the distinction of those that are closely related. These techniques will also be useful to those involved in plant or animal health and medicine, for example in distinguishing a pathogenic strain of a microorganism from one that is non-pathogenic. Furthermore, the present methods will be useful in industrial processes, for example in distinguishing between an organism producing a particular enzyme and an organism not producing the enzyme. In this regard, organisms may be identified and isolated that produce enzymes such as biodegradative enzymes, nucleic acid polymerase or ligase enzymes, amino acid synthetase enzymes, or enzymes involved in carbohydrate fermentation or petroleum product degradation.

It should also be apparent that this method can be used to screen animal tissues to be subsequently used in medical procedures such as tissue or organ transplants, blood transfusions, zygote implantations and artificial inseminations. In such procedures, pre-screening of the subject tissues for the presence of particular polymorphic DNA fragments or genetic markers may improve the success of tissue or organ transplants (by decreasing the likelihood of rejection due to donor-recipient genetic incompatibility) and of zygote implantations (by eliminating the use of genetically defective zygotes). Similarly, use of these methods will reduce the chances of transmission of infectious diseases (e.g., hepatitis and AIDS) in medical procedures that are often prone to such transmission, such as blood transfusions and artificial insemination. Finally, use of the present invention for identification and isolation of unique genomic DNA fragments will assist in forensic science in such applications as crime-scene analysis of blood, tissue and body secretions containing small amounts of DNA, as well as in paternity testing.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

Materials and Methods

The following materials and methods were used for all examples:

Cells and Tissues

Bacterial cells (Agrobacterium tumefaciens, strains K84, A4, 15834, Ag44, AB473, pp-1, Rm1021, C58, AB4, S21, Ag57, AB3, A136, TM4, S4 and 4BA) were obtained from University of Washington (Seattle, Wash.). Fungal cells (Collectotrichum spp., strains Ari-NW, S1-1, 586, 486, 1418(8), 683(Lc), S7RR, C129, 662(10) and C9) were obtained from U.S.D.A. (Beltsville, Md.). Plant tissues (soybean, strains Noric and BARC; peach, strains Bologna Pillar and Flavortop) were obtained from U.S.D.A. (Beltsville, Md.).

Isolation of DNA

Well-separated colonies of different strains of Agrobacterium tumefaciens were individually resuspended in 5 ml of YM broth (0.4 g/L yeast extract, 10 g/L mannitol, 0.1 g/L NaCl, 0.2 g/L MgSO₄.7H₂O, 0.5 g/L K₂HPO₄, pH 7.0). 1.0 ml of cultured cells (incubated at 30° C. for 36-48 h) was transferred into a microcentrifuge tube, and the cell pellets were collected by centrifuging at 11,000×g for 5 min at about 20-25° C. The pellets were resuspended in 1 ml TES buffer (8% sucrose, 50 mM NaCl, 20 mM TRIS®-HCl, 1 mM EDTA, pH 8.0), and incubated at 25° C. for 5 min with 1 mg/ml lysozyme. 100 ul of 10% SDS was then added and the tube was vortexed. The DNA was extracted with phenol-chloroform and precipitated with 0.3 M sodium acetate and 1 volume of isopropanol. The pellet was washed with 70% ethanol and centrifuged at 11,000×g for 10 min at room temperature. The DNA pellet was dissolved in 200 ul of TE buffer (10 mM Tris-HCl, 1 mM EDTA, pH 8.0) and digested with 1 ug/ml RNAse A at 37° C. for 10 min. The phenol-chloroform extraction and isopropanol precipitation were repeated. The final DNA pellet was dissolved in 200 ul of TE buffer.

For genomic DNA isolation from Collectotrichum, spore suspensions were inoculated into Czapek-Dox broth and incubated at 23° C. for eight days on a shaker. The resultant mycelial mass was then collected and stored at −70° C. until being processed. Total DNA was prepared from 1.5 ml cultures according to Rehner and Uecker, Can. J Bot. 72:1666-1674 (1995), and separated on 0.5-0.7% agarose gels in TAE buffer (14 mM Tris-acetate, 1 mM EDTA, pH 8.3). For plant genomic DNA isolation, leaf tissues of soybean and peach were quickly frozen in liquid nitrogen and ground using mortar and pestle, and purified by modified CtBr procedure as described by Lin et al. (FOCUS 16:72-77 (1995)).

AFLP Analysis with ³²P-Labeled Primers

AFLP assays were performed as described previously (Vos, P. et al., Nucl. Acids Res. 23(21):4407-4414 (1995); Lin, J.-J., and Kuo, J., FOCUS 17(2):66-70 (1995)). Briefly, 500 ng genomic DNA was digested with EcoRI and MseI, and the enzymes were inactivated at 70° C. for 15 min. The resulting DNA fragments were ligated with 5 ul of EcoRI and MseI adapters, to form a ligation mixture. For preselective amplification, 5 ul of the ligation mixture, diluted 10-fold with distilled water, was amplified for 20 cycles of 94° C. for 30 seconds, 56° C. for 60 seconds, 72° C. for 60 seconds using EcoRI+0 primer (SEQ ID NO:1) and MseI+0 primer (SEQ ID NO:2) for Agrobacterium and Collectotrichum cells, EcoRI+0 (SEQ ID NO:1) and MseI+G (SEQ ID NO:7) for peach, and EcoRI+A (SEQ ID NO:8) and MseI+C (SEQ ID NO:9) for soybean. For selective amplification, EcoRI primers with 1 to 3 selective nucleotides were labeled with ³²P using T4 polynucleotide kinase. 5 ul of labeled or unlabeled EcoRI primers were mixed with 0.5 ul of preamplified DNA diluted 50-fold with distilled water, PCR buffer, and the MseI primers with 1 to 3 selective nucleotides. The mixtures were amplified for 1 cycle of 94° C. for 30 seconds, 65° C. for 30 seconds, 72° C. for 60 seconds; lowering the annealing temperature by 0.7° C. per cycle for 12 cycles; and then 23 cycles of 94° C. for 30 seconds, 56° C. for 60 seconds and 72° C. for 60 seconds. After adding 20 ul of sequencing loading buffer (98% formamide, 10 mM EDTA, 0.025% xylene cyanol, 0.025% bromophenol blue), the mixtures were heated at 90° C. for 3 minutes, and 2 ul of the samples were electrophoresed in a 5% polyacrylamide sequencing gel containing 8 M urea. The gel was dried and exposed to X-ray film (for ³²P-labeled primers).

AFLP Analysis with Blotting/Hybridization

For AFLP analysis using membrane blotting and hybridization, the amplifications were performed in the same manner as for the radioisotopic version described above, except that the EcoRI primers were not labeled and were diluted with water and not with the components used in the labeling reaction. The MseI primers were diluted as directed above, except that the diluent contained unlabeled EcoRI primers.

After selective amplification was completed, sequencing loading buffers were added to the samples, and the amplified DNA fragments were separated according to size on 5% polyacrylamide sequencing gels using a V-16 gel electrophoresis unit (Life Technologies, Inc; Rockville, Md.). Separated DNA fragments were blotted onto nitrocellulose or nylon membranes, such as Biodyne B (Life Technologies, Inc.; Rockville, Md.), according to standard techniques (Southern, E. M., J. Mol. Biol. 98:503-517 (1975); Watson, J. D., et al., in: Recombinant DNA, 2nd Ed., New York: Scientific American Books, pp. 127-130 (1992)). Briefly, membrane was cut to the size of the gel, pre-wet in electrophoresis buffer, and carefully placed onto the gel surface. After gently removing any residual air bubbles between the gel and the membrane, the membrane was overlaid with 4-5 pieces of filter paper and a glass plate, weighted down, and DNA was allowed to transfer to the membrane over a period of 2-24 hours. Following blotting, the membrane was marked to denote the side containing the blotted DNA fragments, baked for 30-45 minutes at 80° C., and stored dry at about 20-25° C. until use in hybridization reactions.

Hybridization was performed according to standard techniques (Southern, E. M., J. Mol. Biol. 98:503-517 (1975); Watson, J. D., et al., in: Recombinant DNA, 2nd Ed., New York: Scientific American Books, pp. 127-130 (1992)). Briefly, ACES prehybridization/wash (50 mM Na₂HPO₄, 0.1% SDS, pH 7.2) and hybridization buffers (50 mM Na₂HPO₄, 1% Hammerstein casein, 0.1% SDS, pH 7.2) buffers were prewarmed to 42° C., and the membrane comprising the transferred DNA placed DNA side up into a container with 25 ml of the prewarmed ACES prehybridization buffer and incubated for 20 minutes while shaking at a moderate speed. 25 ml of ACES hybridization buffer containing 6.25-12.0 μl of AFLP hybridization probe labeled either with alkaline phosphatase or with ³²P was then added, and the membrane incubated an additional 20 minutes at 42° C. while shaking at a moderate speed. After hybridization, the solution was removed, the membrane was washed twice with 100 ml each of prewarmed ACES prehybridization/wash buffer and incubated for 10 minutes at 42° C. while shaking. 1×ACES 2.0 final wash buffer (10 mM Tris-HCl, 150 mM NaCl, pH 8.6) was prepared by diluting the 10×ACES 2.0 final wash buffer with distilled water, and used to wash the membrane twice for 5 minutes each.

Chemiluminescent detection. Following the final wash, the membrane was removed and placed into a Photogene Development folder (Life Technologies, Inc; Rockville, Md.). 7-10 ml of CDP-Star detection reagent (Tropix, Inc.; Bedford, Mass.) was added to the development folder, and the reagent carefully moved over the membrane for 2-5 minutes. Membrane was then removed, placed into a new Photogene development folder, and exposed to Bio-Max MR film (Kodak; Rochester, N.Y.) for 45 minutes.

Visible Color detection. For color detection, the membrane blotting, prehybridization and hybridization steps were the same as described above for chemiluminescent detection. After the final wash, the membrane was washed once with blocking buffer (0.1 M TRIS®-HCl, 0.1 M NaCl, 50 mM MgCl₂, pH 9.4). Membrane was transferred to a hybridization bag containing 7.5 ml of blocking buffer with 2.5 mg of NBT (Nitroblue tetrazolium) and 1.25 mg of BCIP (5-bromo-4-chloro-3-indolylphosphate), and incubated in the dark for 3 hours or until development of color.

³²P detection. For radioisotopic detection of AFLP, the membrane blotting, prehybridization and hybridization steps were the same as described above for chemiluminescent detection, except AFLP hybridization probes were labeled with ³²P instead of alkaline phosphatase. After the final wash, the dried membrane was exposed to X-ray films to make autoradiograms.

### Example 1

### Detection of Polymorphic DNA Fragments or Genetic Markers by AFLP and Membrane Hybridization

Initially, attempts were made to examine the efficacy of membrane hybridization in the detection of AFLP-defined polymorphic DNA fragments or genetic markers. Genomic DNA was prepared from various strains of Agrobacterium tumefaciens, subjected to AFLP using primers that were either ³²P-labeled (“traditional AFLP”) or unlabeled (“membrane hybridization AFLP”), and the resulting AFLP-defined DNA fragments were then separated on sequencing gels. Gels containing fragments defined using radiolabeled primers were then dried and used to prepare autoradiograms, while those using unlabeled primers were blotted onto nylon membranes, and the blots probed via stringent hybridization with alkaline phosphatase-labeled oligonucleotide probes. Polymorphic DNA fragments were then identified via autoradiography (for ³²P-labeled primers) or chemiluminescence (for membrane hybridization with alkaline phosphatase-labeled probes), and the resulting patterns between the two techniques compared.

As shown in FIGS. 1A and 1B, the membrane hybridization methodologies of the present invention (FIG. 1B) are approximately as sensitive in detecting strain-specific polymorphic DNA fragments, or potential genetic markers, as is the traditional AFLP approach using radiolabeled primers (FIG. 1A). While there were some apparent differences in intensities of the bands between the two techniques (see, e.g., arrows in lanes 8-10), the membrane hybridization AFLP approach appeared to be as sensitive as, and provided similar results to, the traditional radiolabeled primer AFLP approach in detecting polymorphic DNA fragments in the various Agrobacterium strains. These results indicate that polymorphic DNA fragments or genetic markers may be identified using AFLP and the membrane hybridization approaches of the present invention. Furthermore, these approaches may be used to distinguish between different strains of an organism, including between pathogenic (e.g., C58) and nonpathogenic (e.g., A136) strains.

### Example 2

### Range of Genome Sizes Resolvable by AFLP and Hybridization

Having demonstrated the efficacy of membrane hybridization in defining polymorphic DNA fragments or genetic markers via AFLP, the range of genome sizes that could be resolved by the present methods was examined. For these studies, samples of genomic DNA were prepared from soybean (genome size of 1×10⁹ base pairs (“bp”)) and Collectotrichum spp. (genome size of 5×10⁷ bp) were prepared and analyzed by the AFLP membrane hybridization approach outlined in Example 1.

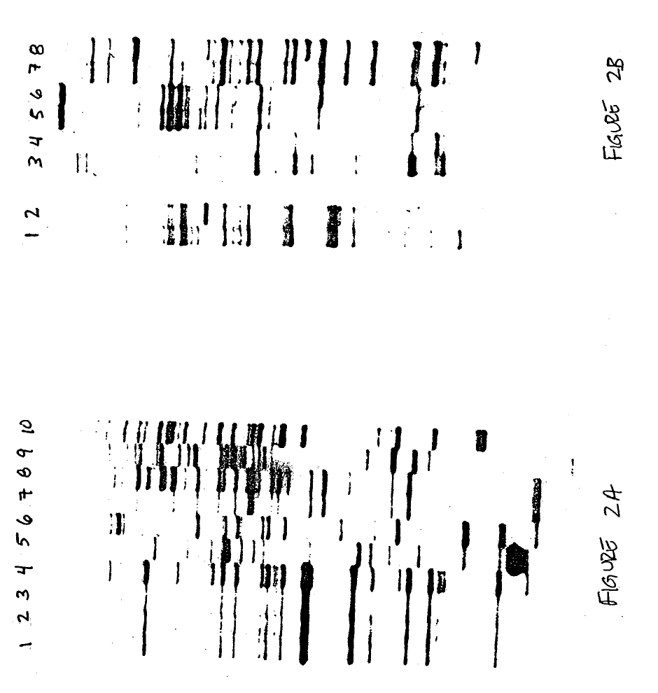

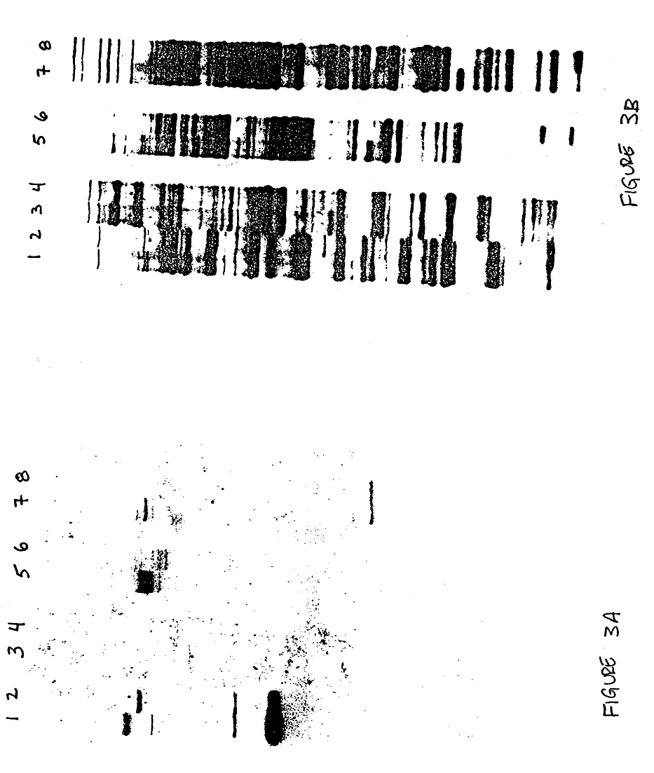

As shown in FIGS. 2A and 2B, the present methods are suitable for detecting polymorphic DNA fragments in a wide range of genomes, including a relatively small genome (Collectotrichum; FIG. 2A) and a larger genome (soybean; FIG. 2B). Coupled with the findings that the present methods are suitable for resolving polymorphic DNA fragments in Agrobacterium (genome size of 5×10⁶ bp; FIG. 1B) and peach (genome size of 3×10⁸ bp; FIG. 3B), these results indicate that the membrane hybridization AFLP methods of the present invention can resolve polymorphic DNA fragments or genetic markers over at least a 200-fold range in genome size (i.e., Agrobacterium to soybean).

### Example 3

### Detection of Known DNA Polymorphisms or Genetic Markers

To explore the utility of the methods of the present invention in detecting known polymorphic sequences or genetic markers in a sample of DNA, AFLP with membrane hybridization or with labeled amplification probes was used to examine plant DNA samples. For these studies, genomic DNA was purified from two different varieties of peach (Bologna Pillar and Flavortop; U.S.D.A., Beltsville, Md.). Some samples were subjected to AFLP using radiolabeled primers and detection by autoradiography as described (Lin, J.-J., and Kuo, J., FOCUS 17(2):66-70 (1995)), while others were amplified by AFLP with unlabeled probes, blotted onto membranes and probed with telomere repeat sequence (TTTAGGG)₃ oligonucleotides.

As shown in FIG. 3, the combination of AFLP with membrane hybridization using telomere probes rapidly and sensitively detected polymorphisms or genetic markers between different strains of peach (FIG. 3A). Conversely, these polymorphisms were far more difficult to discern using the radiolabeled primer approach with AFLP (FIG. 3B), which instead delineated other polymorphisms between the strains. These results demonstrate that the combination of AFLP with membrane blotting and hybridization is able to detect subtle polymorphisms or potential genetic markers, such as the presence of different telomere repeats, between different DNA samples, even in those obtained from closely related individuals (e.g., strains or varieties of a species). Since telomere repeat sequences are associated with cellular carcinogenesis and aging (Sharma, H. W., et al., Anticancer Res. 16(1):511-515 (1996); Vaziri, H., and Benchimol, S., Exp. Gerontol. 31(1-2):295-301 (1996); Smith, J. R., and Pereira-Smith, O. M., Science 273:63-67 (1996); Shay, J. W., et al., Leukemia 10(8):1255-1261 (1996)), the present methods may provide a sensitive means of detecting tumor cells and senescent cells, which will be useful in diagnosis of various cancers and degenerative diseases.

Attempts to detect telomere sequences have traditionally used Southern blot hybridization with telomere repeat oligonucleotide probes, a technique which results in the generation of smeared and otherwise poorly resolved bands perhaps due in part to the presence of only low quantities of the target sequence. By combining AFLP with membrane hybridization, the present invention overcomes these problems—the target sequences, or DNA fragments comprising them, are amplified to a level where they can be detected using various hybridization probes without smearing on the gels. Moreover, using the present approaches the membrane may be washed to remove the hybridized probe and then re-examined with a different probe (e.g., oligonucleotides, cDNAs, genomic DNA samples, restriction fragments, minisatellite sequences, etc.). This washing and reprobing may be carried on for a number of cycles, generating a set of results for a battery of target sequences, known and unknown, thus providing the ability to identify and characterize numerous polymorphisms and potential polymorphic DNA fragments in a sample of DNA.

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is clamed is:

1. A method for identifying a polymorphic DNA fragment or a genetic marker from a first sample of DNA, said polymorphic DNA fragment or genetic marker not being present in a second sample of DNA, said method comprising:
(a) digesting a first and second samples of DNA with at least one restriction enzyme to give a collection of restriction fragments;
(b) amplifying said collection of restriction fragments;
(c) separating said restriction fragments according to size;
(d) immobilizing said separated restriction fragments on a solid support;
(e) contacting said immobilized restriction fragments with one or more hybridization probes complementary to one or more of said immobilized restriction fragments under conditions stringent for hybridization of said hybridization probes to said immobilized restriction fragments; and
(f) comparing the pattern of restriction fragments from said first sample of DNA to the pattern of fragments from said second sample of DNA.

2. A method for identifying a polymorphic DNA fragment or a genetic marker from a second sample of DNA, said polymorphic DNA fragment or genetic marker not being present in a first sample of DNA, said method comprising:
(a) digesting a first and second samples of DNA with at least one restriction enzyme to give a collection of restriction fragments;
(b) amplifying said collection of restriction fragments;
(c) separating said restriction fragments according to size;
(d) immobilizing said separated restriction fragments on a solid support;
(e) contacting said immobilized restriction fragments with one or more hybridization probes complementary to one or more of said immobilized restriction fragments under conditions stringent for hybridization of said hybridization probes to said immobilized restriction fragments; and
(f) comparing the pattern of restriction fragments from said second sample of DNA to the pattern of fragments from said first sample of DNA.

3. The method of claim 1 or claim 2, wherein said solid support is a nylon membrane or a nitrocellulose membrane.

4. The method of claim 1 or claim 2, further comprising removing said oligonucleotide from said solid support; contacting said immobilized restriction fragments with a second oligonucleotide complementary to one or more of said immobilized restriction fragments under conditions stringent for hybridization of said oligonucleotide to said immobilized restriction fragments; and comparing the pattern of restriction fragments from said second sample of DNA to the pattern of fragments from said first sample of DNA.

5. The method of claim 1 or claim 2, further comprising isolating said polymorphic DNA fragment or genetic marker and inserting said polymorphic DNA fragment or genetic marker into a vector.

6. The method of claim 5, wherein said polymorphic DNA fragment or genetic marker is amplified prior to insertion into said vector.

7. The method of claim 1 or claim 2, further comprising sequencing said polymorphic DNA fragment or genetic marker.

8. A method for isolating a polymorphic DNA fragment or genetic marker from a first sample of DNA, said method comprising:
(a) mixing a polymorphic DNA fragment or genetic marker identified according to claim 1 or claim 2, or an oligonucleotide probe complementary to said polymorphic DNA fragment or genetic marker, with a first sample of DNA under conditions stringent for hybridization of said polymorphic DNA fragment or genetic marker, or of said oligonucleotide probe, to said first sample of DNA; and
(b) isolating a DNA molecule which is complementary to said polymorphic DNA fragment or genetic marker, or to said oligonucleotide probe.

9. The method of claim 8, wherein said isolation is accomplished by a method selected from the group of methods consisting of gel electrophoresis, density gradient centrifugation, sizing chromatography, affinity chromatography, immunoadsorption, and immunoaffinity chromatography.

10. The method of claim 8, further comprising sequencing said isolated polymorphic DNA fragment or genetic marker.

11. The method of claim 8, further comprising amplifying said isolated polymorphic DNA fragment or genetic marker.

12. The method of claim 8, further comprising inserting said isolated polymorphic DNA fragment or genetic marker into a vector.

13. The method of claim 12, wherein said vector is an expression vector.

14. The method of claim 1 or claim 2, wherein said amplification is accomplished by a method comprising ligating one or more adapter oligonucleotides to said unique restriction fragments to form a DNA-adapter complex; hybridizing said DNA-adapter complex, under stringent conditions, with one or more oligonucleotide primers which are complementary to said adapter portion of said DNA-adapter complex to form a hybridization complex; and amplifying said DNA-adapter complex.

15. The method of claim 14, further comprising inserting said DNA-adapter complex into a vector.

16. The method of claim 1 or claim 2, wherein said first sample of DNA and said second sample of DNA are derived from a source selected from the group consisting of an individual cell, a tissue, an organ, and a whole organism.

17. The method of claim 16, wherein said cell is a prokaryotic cell.

18. The method of claim 16, wherein said cell is a eukaryotic cell.

19. The method of claim 16, wherein said tissue is an animal tissue.

20. The method of claim 19, wherein said animal tissue is a human tissue.

21. The method of claim 20, wherein said human tissue is a human embryonic tissue.

22. The method of claim 20, wherein said human tissue is a human fetal tissue.

23. The method of claim 16, wherein said tissue is a plant tissue.

24. The method of claim 1 or claim 2, wherein said polymorphic DNA fragment or genetic marker is selected from the group consisting of a cancer marker, an infectious disease marker, a genetic disease marker, a marker of embryonic development, a tissue-specific marker and an enzyme marker.

25. The method of claim 1 or claim 2, wherein said first sample of DNA is derived from a sample of an animal suffering from cancer and said second sample of DNA is derived from a animal not suffering from cancer.

26. The method of claim 1 or claim 2, wherein said first sample of DNA is derived from a cancerous animal tissue and said second sample of DNA is derived from a noncancerous animal tissue.

27. The method of claim 26, wherein said first sample and said second sample are derived from the same animal.

28. The method of claim 1 or claim 2, wherein said first sample of DNA is derived from an animal suffering from a genetic disease and said second sample of DNA is derived from an animal not suffering from said genetic disease.

29. The method of claim 1 or claim 2, wherein said first sample of DNA is derived from a diseased plant and said second sample of DNA is derived from a non-diseased plant.

30. The method of claim 1 or claim 2, wherein said first sample of genomic DNA is derived from a plant resistant to an environmental stress and said second sample of genomic DNA is derived from a plant not resistant to said environmental stress.

31. The method of claim 30, wherein said environmental stress is selected from the group consisting of drought, excess temperature, diminished temperature, chemical toxicity by herbicides, pollution, excess light and diminished light.

32. The method of claim 1 or claim 2, wherein said first sample of DNA is derived from a pathogenic microorganism and said second sample of DNA is derived from a nonpathogenic microorganism.

33. The method of claim or claim 2, wherein said first sample of DNA is derived from a organism producing an enzyme and said second sample of DNA is derived from an organism not producing said enzyme.

34. The method of claim 33, wherein said enzyme is a restriction enzyme, an enzyme degrading a petroleum product, a biodegradative enzyme, a nucleic acid polymerase enzyme, a nucleic acid ligase enzyme, an amino acid synthetase enzyme or an enzyme involved in carbohydrate fermentation.

35. A method of determining the relationship between a first individual and a second individual, said method comprising:
(a) digesting samples of genomic DNA obtained from said first and second individuals with at least one restriction enzyme to give a collection of restriction fragments;
(b) amplifying said collection of restriction fragments;
(c) separating said restriction fragments from said first and said second individual according to size;
(d) immobilizing said separated restriction fragments on a solid support;
(e) contacting said immobilized restriction fragments with one or more hybridization probes complementary to one or more of said immobilized restriction fragments under conditions stringent for hybridization of said hybridization probes to said immobilized restriction fragments; and
(f) determining the similarities and dissimilarities of the sizes or concentrations of the restriction fragments separated in (c).

36. The method of any one of claims 1, 2 or 35, wherein said oligonucleotide comprises a telomere repeat sequence.

37. The method of any one of claims 1, 2 or 35, wherein said hybridization probes are distinctly and detectably labeled.

38. A kit for the identification and isolation of a polymorphic DNA fragment or genetic marker comprising two or more containers, wherein a first container contains an oligonucleotide adapter DNA molecule and a second container contains a DNA polymerase.

39. The kit of claim 38, wherein said DNA polymerase is a thermostable DNA polymerase.

40. The kit of claim 39, wherein said thermostable DNA polymerase is selected from the group consisting of Taq DNA polymerase, Tne DNA polymerase, Tma DNA polymerase, and mutants and derivatives thereof.

41. The kit of claim 38, further comprising one or more additional containers containing an oligonucleotide hybridization probe or a restriction endonuclease.

42. The kit of claim 41, wherein said oligonucleotide hybridization probe is detectably labeled.
